(19) United States
(12) Patent Application Publication
(10) Pub. No.: US 2025/0207192 A1
(43) Pub. Date: Jun. 26, 2025

(54) TARGETED ENRICHMENT OF LARGE DNA MOLECULES FOR LONG-READ SEQUENCING USING FACS OR MICROFLUIDIC PARTITIONING

(71) Applicant: SAMPLIX APS, Birkerød (DK)

(72) Inventors: Thorarinn BLONDAL, Birkerød (DK); Peter MOURITZEN, Copenhagen V (DK)

(73) Assignee: SAMPLIX APS, Birkerød (DK)

(21) Appl. No.: 18/849,453

(22) PCT Filed: Mar. 16, 2023
(86) PCT No.: PCT/DK2023/050043

§ 371 Date: Sep. 20, 2024

(30) Foreign Application Priority Data
Mar. 21, 2022 (DK) PA202200230

(51) Int. Cl. C12Q 1/6874; C12Q 1/6806

(52) U.S. Cl. CPC C12Q 1/6874; C12Q 1/6806

(57) ABSTRACT

Provides a method to obtain enrichment of large DNA molecules for long-read sequencing using FACS or microfluidic partitioning. Furthermore, the present invention relates to a kit comprising a plurality of 5 microfluidic devices and a plurality of fluids configured for use with the system and the method.

### TECHNICAL FIELD

The present invention relates to an in vitro method for enrichment of large DNA molecules to be used in long-read DNA sequencing wherein the DNA molecules comprise a known nucleotide sequence element.

The present invention further relates to a kit and a system for performing the in vitro method for enrichment of large DNA molecules.

### BACKGROUND

The high precision, specificity and efficiency of CRISPR has provided an unprecedented improvement in genetic engineering relative to former technologies for gene targeting. This improvement has accelerated and extended the exploitation of genetic engineering in organisms from plants and animals to humans. The accuracy of CRISPR editing enables precise modifications such as insertion of a wildtype gene to replace a mutated disease-causing gene, where this approach is being investigated to treat SCID, cystic fibrosis, and sickle cell disease. However, the application of CRISPR for gene therapy in humans has spurred concerns about safety where the most pressing relate to the accuracy of the applied CRISPR editing and risk of potential off-target editing (Blondal et al., 2021). Other therapeutic treatments make use of viral vectors to integrate ‘cargo’ DNA into the genome of a cell, such as the use of lentivirus to deliver CAR cassettes in CAR-T therapy. Since the integration is semi-random there is an inherent risk of CAR cassettes integrating near oncogenes and suppressors. The genetic modification with virally integrating vectors or with CRISPR together with donor DNA is performed on a population of cells. Since not all cells are modified, the outcome of such genetic modifications results in a heterogeneous cell population. Cells modified using virally integrating vectors may differ in the number of integrated copy numbers per cell and in the location of integration(s) in the genome.

For the development of engineered cell therapy for a specific disease, it is essential (and even an FDA and EMA requirement) that gene editing outcome can be fully characterized and the desired editing verified. This requires long-read sequencing in order to determine the long context information of the DNA modification(s) made in individual cells, which is costly since ultra-deep sequencing is needed to provide sufficient sequence coverage per single cell genome. Two methods for target enrichment are CRISPR-based enrichment Steele, et al., (2020) and Xdrop-targeted enrichment (Blondal et al., 2021), which each facilitate targeted long-read sequencing, providing significantly more cost effective verification of gene editing outcome.

However, with the increasing use of gene editing technologies/therapies there is a continuing need to develop improved tools to characterise and verify the long-context information of DNA modification(s) made in individual cells and cell populations.

### SUMMARY OF THE INVENTION

According to a first aspect, the invention provides an in vitro method for enriching for one of more target DNA molecules being DNA molecules comprising a specific motif or sequence from a sample of mixed DNA molecules, wherein the method comprises the steps of:

Thus, according to a first aspect of the present invention, there is provided an in vitro method for enriching for one of more target DNA molecules being DNA molecules comprising a specific motif or sequence from a sample of mixed DNA molecules, wherein the method comprises the steps of:
a) providing a liquid sample of mixed DNA molecules comprising one or more specific target DNA molecule,
b) fragmenting the mixed DNA molecules of the liquid sample to obtain a population of DNA molecules having an average size of from 5 to 40 kb,
c) ligating adaptors to the ends of the population of DNA molecules of step (b) to obtain a liquid sample of adaptor-ligated DNA molecules,
d) forming of an emulsion of a multiple of double emulsion droplets from the liquid sample obtained in step (c),
e) specifically detecting droplets containing at least one of said target DNA molecules,
f) physically sorting and coalescing droplets containing at least one of said target DNA molecules, and
g) general amplification of the adaptor-ligated DNA molecules of the selected and coalesced droplets obtained in step (f).

According to a second aspect, the invention provides a kit of parts for performing the in vitro method according to the first aspect, comprising:
i) one or more microfluidic devices (cartridges) to form to form the double-emulsion droplets of step d) and optionally the second emulsion of step (g),
ii) adaptors suitable to perform step c) of ligating adaptors to the ends of the population of DNA molecules,
iii) vials of a suitable oil composition comprising a suitable surfactant and the necessary buffers to form the emulsions of step d) and optionally of the second emulsion of step (g),
iv) vials of a suitable breakage solution and a suitable buffer/dye to rescue the DNA in the droplets selected in step f), and optionally after the general amplification performed on the second emulsion composition, and
v) a manual for performing the method.

The present invention relates to different aspects including the devices and methods described above and in the following. Each aspect may yield one or more of the benefits and advantages described in connection with one or more of the other aspects. Each aspect may have one or more embodiments with all or just some of the features corresponding to the embodiments described in connection with one or more of the other aspects and/or disclosed in the appended claims.

Other systems, methods and features of the present invention will be or will become apparent to one having ordinary skill in the art upon examining the following drawings and detailed description. It is intended that all such additional systems, methods, and features be included in this description, be within the scope of the present invention and protected by the accompanying claims.

### BRIEF DESCRIPTION OF THE DRAWINGS

FIG. 1. Schematic illustration of one embodiment of the present method wherein the general amplification step (VIII) is a long-range PCR (LR-PCR) performed in bulk, wherein 1 is the PCR-adaptor; 2 one of the two primers for the specific gene region used to perform the region-specific enrichment; 3 a positive green fluorescent droplet; 4) is a sample of positive green fluorescent droplets obtained by the sorting. Where the steps of the method are: I) providing a liquid sample of mixed DNA molecules; II) fragmenting the mixed DNA molecules of the liquid sample; III) ligating adaptors to the ends of the population of DNA molecules; VI) forming of an emulsion of a multiple of double emulsion droplets; V) perform the PCR reaction to specifically detect droplets; VI) physically sorting droplets; VII) coalescing selected droplets; VIII) general amplification of the adaptor-ligated DNA molecules; IX) perform the nanopore sequencing of amplified adaptor-ligated DNA molecules.

FIG. 2. A schematic illustration of a method wherein the general amplification step is a standard dMDA amplification (multiple displacement amplification performed in droplets). I) Providing a liquid sample of mixed DNA molecules; II) forming of an emulsion of a multiple of double emulsion droplets; III) specifically detecting droplets; IV) physically sorting droplets; V) coalescing droplets; VI) forming of an emulsion of a multiple of single emulsion droplets; VII) general amplification of the selected DNA molecules; VIII) coalescing droplets; and IX) perform the nanopore sequencing.

FIG. 3A schematic illustration of one embodiment of the present method as shown in FIG. 1, wherein the general amplification step (VIII) is a LR-PCR and is performed in an emulsion of double-emulsion droplets. Reference number 1 is the PCR-adaptor; 2 one of the two primers for the specific gene region used to perform the region-specific enrichment; 3 a positive green fluorescent droplet; and 4 is a sample of positive green fluorescent droplets obtained by the sorting.

The steps of the method are: I) providing a liquid sample of mixed DNA molecules; II) fragmenting the mixed DNA molecules of the liquid sample; III) ligating adaptors to the ends of the population of DNA molecules; IV) forming of an emulsion of a multiple of double emulsion droplets; V) perform the PCR reaction to specifically detect droplets; VI) physically sorting droplets; VII) coalescing droplets; VIII) forming of an emulsion of single or double emulsion droplets; XI) general amplification of the adaptor-ligated DNA molecules; X) coalescing droplets; IX) perform the nanopore sequencing.

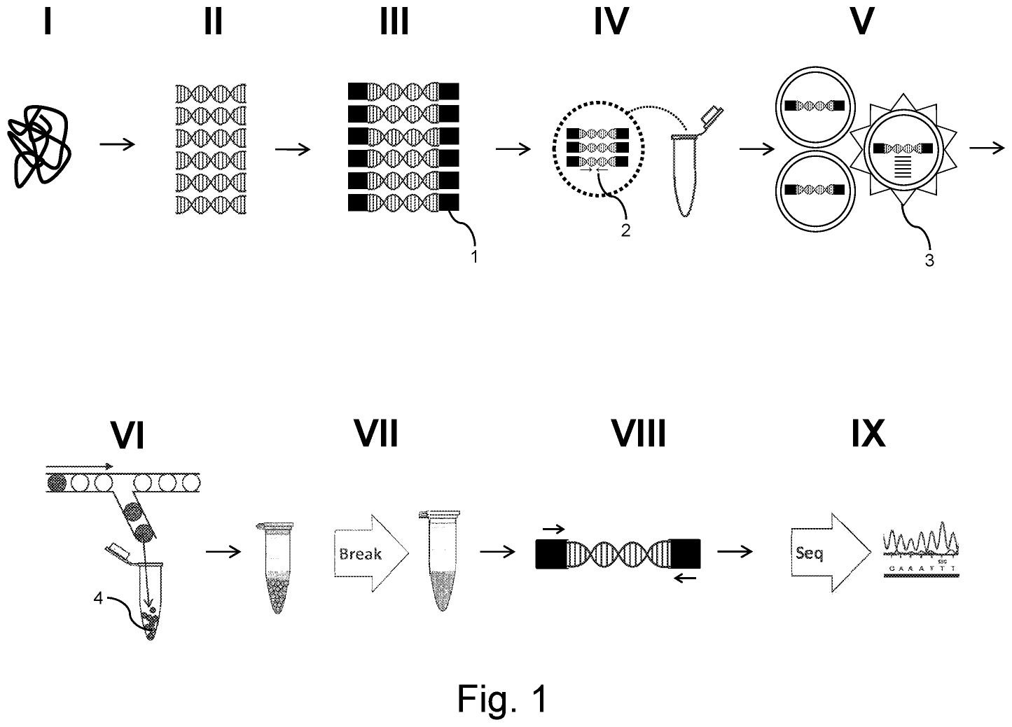

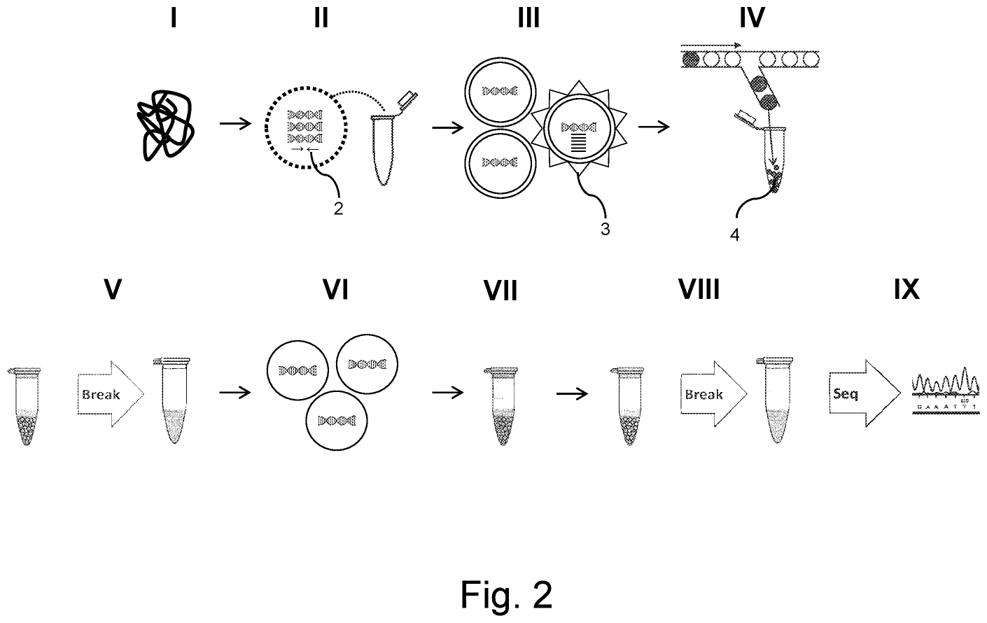

FIG. 4. An illustration of DNA sequence data obtained for [A]: amplified DNA molecules prepared according to the method shown in FIG. 2, wherein the general amplification step was performed by dMDA in single emulsion droplets; [B]: amplified DNA molecules prepared according to the method shown in FIG. 1, wherein the general amplification step was LR-PCR performed in bulk; and [C] wherein the sequence data in [A] is superimposed on [B]. Each of [A]. [B] and [C] show the number of bases in the primary mapped reads as a function of the length of the read (binsize of histogram is 500 bases).

FIG. 5. An illustration of DNA sequence data obtained amplified DNA molecules prepared according to the method shown in FIG. 1, wherein the general amplification step was LR-PCR performed in bulk; wherein [A] shows the number of bases in primary mapped reads as a function of the length of the read (binsize of histogram is 500 bases), [B] shows the number of bases in the mapped part of the primary mapped reads as a function of the length of the read (binsize of histogram is 500 bases); and [C] wherein the sequence data in [B] is superimposed on [A].

FIG. 6. An illustration of DNA sequence data obtained amplified DNA molecules prepared according to the method shown in FIG. 2, wherein the general amplification step was performed by dMDA in single emulsion droplets; wherein [A] shows the number of bases in primary mapped reads as a function of the length of the read (binsize of histogram is 500 bases), [B] shows the number of bases in the mapped part of the primary mapped reads as a function of the length of the read (binsize of histogram is 500 bases); and [C] wherein the sequence data in [B] is superimposed on [A].

FIG. 7. An illustration of DNA sequence data wherein the data in FIG. 6C superimposed on the data of FIG. 5C. Binsize is 500 bases.

FIG. 8. An illustration of DNA sequence data obtained for [A]: amplified DNA molecules prepared according to the method shown in FIG. 2, wherein the general amplification step was performed by dMDA in single emulsion droplets; [B]: amplified DNA molecules prepared according to the method shown in FIG. 1, wherein the general amplification step was LR-PCR performed in bulk; and [C] wherein the sequence data in [A] is superimposed on [B]. Each of [A]. [B] and [C] show the ratio of number of bases in the aligned part of the primary mapped read relative to the number of bases in the primary mapped read as a function of the length of the primary read (binsize of histogram is 500 bases).

### DEFINITIONS

Prior to a discussion of the detailed embodiments of the invention a definition of specific terms related to the main aspects of the invention is provided.

The term “adaptor-ligated DNA molecules” as used herein refer to DNA molecules with adaptors ligated to the two ends of the DNA molecules.

The term “Align/aligning” as used herein describe arranging the two sequences of DNA or RNA to identify regions of similarity. Typically the similarity being identified is assigned an alignment score.

The term “Alignment score” as used herein is a metric that indicates how similar a read (a sequence) is to the reference.

The term “amplification” as used herein refer to a reaction that form multiple copies of at least one segment of a template DNA molecule

The term “Basecalled bases” as used herein refer to a sequence of bases obtained by transforming electrical signals in a nanopore sequencing device to nucleotide-sequence information. Basecalling is usually the initial step to analyze nanopore sequencing signals. A basecaller translates raw signals (referred to as squiggle) into nucleotide sequences and feeds the nucleotide sequences to downstream analysis.

“Passed basecalled bases” means that the interpretation of the of the electrical signals in sequencing device passed a quality test allowing to assign the signal to the bases.

The term “bases in the aligned part of the primary mapped reads” as used herein refer to the number of aligned bases in the primary reads of a certain range of lengths (bin-length).

The term “bases in the primary mapped reads” as used herein refer to the number of passed basecalled bases in the primary reads of a certain range of lengths.

The term “blunt DNA molecule” as used herein refer to double-stranded DNA molecules with flushed or non-staggered ends as opposed to double-stranded DNA molecules with 3′ or 5′ overhanging ends.

The term “Chimeras” as used herein are sequence artifacts introduced by phi29 DNA polymerase during Multiple Displacement Amplification. Chimeras (i.e. chimeric reads) are the result of alterative secondary structures, that occur in the highly branched DNA, formed during the MDA processing. It appears as DNA rearrangements in the amplified DNA.

The term “coalescing droplets” as used herein refer to the process of destabilising an emulsion of droplets to obtain a non-emulgated two-phase system.

The term “dMDA” as used herein refer to the multiple displacement amplification (MDA) technique described by Blanco et al (1989) and Zanoli et al (2013) but performed in droplets.

The term “double emulsion” as used herein refer to an emulsion predominantly composed of double emulsion droplets and a varying number of oil droplets.

The term “double emulsion droplet” refers to a water-in-oil-in-water droplet (also named w/o/w droplet) and consists of an aqueous droplet inside an oil droplet, i.e., an aqueous core and an oil shell, surrounded by an aqueous carrier fluid.

Preferably, the double emulsion is a monodispersed emulsion, i.e., an emulsion comprising droplets of approximately the same volume. Typically, the w/o/w droplet has a volume of less than 1000 pL, preferably of less than 100 pL. Preferably, a w/o/w droplet has a volume ranging from 0.1 pL to 50 pL, more preferably from 0.25 pL to 25 pL, even more preferably from 0.5 pL to 10 pL, and in particular from 1 pL to 5 pL.

The term “droplet” as used herein refers to a small volume of liquid, typically in a spherical shape, surrounded by an immiscible fluid such as a continuous phase of emulsion. Throughout the present disclosure, the terms “droplet” and “micro-droplet” may be used synonymously. Typically, the droplet has a volume of 1 uL or less, preferably of 1 nL or less, e.g., 0.0001 nL to 1 nL. Single emulsion droplets are usually larger than double emulsion droplets.

There are more methods and devices for forming single- or double-emulsion droplets. Two particularly relevant approaches to form either single- or double-emulsion droplets are described in PCT/EP2020/052409 and PCT/EP2020/052400. In both PCT/EP2020/052409 and PCT/EP2020/052400 the systems for generating droplets imply a single-use cartridge and an instrument all of which are currently marketed and commercially available from Samplix Aps, Herlev, Denmark. The Xdrop instrument (item #IN00100, Samplix ApS, Herlev, Denmark) is designed to perform this task in combination with either the single-emulsion generating cartridge (Samplix 25 item #CA20100) or the double-emulsion generating cartridge (Samplix item #CA10100).

The term “FACS” is short for fluorescence-activated cell sorter.

The term “fluorophore-labelled probes” as used herein refer to a nucleotide-probe sequence with a fluorophore attached to it e.g., Molecular Beacons or Taqman-probes.

The term “general amplification” as used herein is used to describe an amplification process directed to amplify all DNA molecules of a mixed collection of DNA molecules as opposed to a specific amplification.

The terms “adaptor” or “ligating adaptor” refer to a specially designed DNA sequence, which can be recognized as a start site for primer-facilitated DNA strand synthesis, eg. PCR, after ligated at the two ends of a DNA molecule.

The term “long range PCR” is used to describe the PCR amplification of DNA molecules that are 2 kb or more.

The term “Map/mapping” as used herein refer to the process of aligning reads to a reference genome. A mapping program or “mapper” (e.g. the minimap2 program), takes as input a reference genome and the reads. It aims to align each read to the reference genome, allowing for mismatches, indels and softclipping (at the beginning and end of the read). The mapper calculates an alignment score based on matches (usually the longer the stretch of matches, the better the score), and penalizes for introduction of mismatches and indels. The mapper also calculates a mapping score based on how confident it is that the read comes from the reported position.

The term “Mapping score” as used herein is a metric that indicates how confident the “mapper” is that the read comes from the reported position.

The term “MDA” as used herein refer to the Multiple Displacement Amplification technique is a method for amplifying linear DNA (e.g. human DNA) in a cascading, strand displacement reaction that is catalysed by φ29 DNA polymerase in the presence of random hexamer primers, that finds use in whole genome amplification.

The term “microfluidic” implies that at least a part of the respective device/unit comprises one or more fluid conduits being in the microscale, such as having at least one dimension, such as width and/or height, being smaller than 1 mm and/or a cross-sectional area smaller than 1 mm². The smallest dimension, such as a height or a width, of at least one part of the fluid conduit network, such as a conduit, an opening, or a junction, may be less than 500 μm.

The term “microfluidic device”, “microfabricated device” and “cartridge” are used synonymously. It refer to a droplet-forming device which comprises a microfluidic network and which can be used to produce an emulsion of droplets when fitted into a suitable instrument provided with suitable fluids and subjected to conditions which facilitates flow through the microfluidic network of the microfluidic device.

The term “microfluidic sorting device” as used herein refer to a system which comprises a microfluidic network that is able to sort a suspension of particles/droplets.

The term “apex” is used to describe the maximum of a distribution assuming that the distribution can be approximated with a continuous unimodal distribution.

The term “apex of the distribution of the primary reads” as used herein refer to the distribution of the number of passed basecalled bases in the primary reads as a function of read lengths when the distribution is approximated with a continuous unimodal distribution. The “mode of the distribution of the primary reads” is the approximated mode of this continuous unimodal distribution.

The term “number of bases in the aligned part of the primary mapped read” is the sum of bases found in the aligned part of the mapped read. I.e. the sum of bases found in primary mapped reads without the softclipped parts.

The term “number of bases of the primary mapped read” is the sum of bases found in primary aligned reads.

The term “number of reads primary mapped” as used herein is the sum of reads that the “mapper” has designated as primary mapped to the reference.

The term “oil”, “emulsion oil” and “carrier fluid” may be used synonymously in the case of single emulsion droplets. In case of double emulsion droplets, the carrier fluid is typically an aqueous fluid.

The term “PCR” as used herein refer to the Polymerase Chain Reaction technique e.g., as described in U.S. Pat. No. 4,683,195.

The term “Percentage of aligned bases” is the number of bases in the aligned part of the primary mapped read ×100 divided by the number of bases of the primary mapped read.

The term “Percentage of reads primary mapped to reference” as used herein refer to number of primary mapped reads divided by total number of reads.

The term “physically sorting of droplets” as used herein refer to the process wherein droplets containing target DNA molecules are detected and physically selected e.g. by their fluorescence. Subsequently the droplets are sorted into at least two different streams; one stream for the positive droplets and one stream for the negative droplets.

The term “primary read” as used herein refers to un-edited reads that align and map to the reference sequence. Primary reads comprise of the primary aligned part, which aligns to the reference with the highest alignment score at the highest mapping score position, and softclipped parts, which do not align to the reference at this mapping position.

The term “raw read” as used herein refer to unedited passed basecalled base information obtained from an Oxford Nanopore Technologies or similar sequencing device. There is no alignment or mapping information associated with the raw read, i.e. it is not known if the read aligns to a reference.

The term “Read” as used herein is an inferred sequence of bases corresponding to the sequence of a DNA molecule.

The term “Reagent” as used herein refers to a compound or a set thereof, and/or a composition, which is associated to a sample to perform a specific test on the sample. For example, the reaction reagent may be an amplification reagent, specifically, a primer for amplifying a target nucleic acid, a probe and/or a dye for detecting an amplified product, a polymerase, a nucleotide (e.g., dNTP), a magnesium ion, a potassium chloride, a buffer, or any combination thereof.

The term “Reference sequence” as used herein refers to a known nucleotide sequence to which reads are aligned.

The term “Sample” as used herein refers any liquid volume containing a number of DNA molecules. For example, a sample may be a biological sample, such as a biological fluid, a biological entity or an extract of any such items. Examples of the biological fluid include urine, blood, plasma, serum, saliva, semen, faeces, sputum, cerebrospinal fluid, tear fluids, mucus, amniotic fluid, and the like. The biological entity refers to a cell or collection of cells, including bacteria and virus.

The term “sample of mixed DNA molecules” as used herein refers to any liquid volume containing a number of non-identical DNA molecules.

The term “Secondary read” is a read which comprise a “secondary aligned part” i.e. an aligned part of a read that is characterised by a lower alignment score than the primary aligned part.

The term “Sequence”, “DNA-sequence”, “polynucleotide” or “nucleic acid-sequence,” is used interchangeably herein, it refers to a polymeric form of deoxyribonucleotides of any length.

The term “shearing orifice” as used herein refers to the orifice in a DNA shearing device such as the Covaris g-TUBE's (Covaris, LLC. Woburn, Massachusetts)

The term “single emulsion/single emulsion droplets” as used herein refer to an emulsion predominantly composed of single emulsion droplets.

The term “single emulsion droplet” refers to an isolated portion of an aqueous phase that is completely surrounded by a non-aqueous carrier fluid.

The term “softclipped parts” as used herein refers to the part(s) of a primary read that do not align to the reference sequence

The term “specific detection of droplets” as used herein refers to a process wherein droplets containing at least one target DNA molecule may be “specifically detected” by the presence of the target DNA sequence e.g. determined by PCR including qPCR, by hybridization based assays or by assays detecting an RNA or protein product of the target sequence. Typically, droplets containing the target DNA molecule are reacted (e.g. stained) to fluoresce when excited by UV light.

The term “suitable oil” may be any type of carrier fluid which is sufficiently immiscible with water to be able to form a water-oil emulsion of aqueous droplets. The carrier fluid can be a non-polar solvent, decane, fluorocarbon oil, silicone oil or any other oil (for example mineral oil). A fluorocarbon oil is preferred, e.g. Novec HFE-7500 (Cas. no. 297730-93-9), 3M Co., Maplewood, MN, USA.

The term “suitable surfactant” as used herein refer to surfactants that serve to stabilize emulsions derived from two or more immiscible liquids. Fluorosurfactants are preferred for stacilizing aqueous droplets dispersed in a fluorophilic continuous phase (e.g. single-emulsion droplets), or aqueous droplets, each encapsulated within a droplet of fluorophilic liquid, that are dispersed in a bulk aqueous continuous phase (e.g. double-emulsion droplets). Fluorosurfactants are typically comprised of a fluorophilic tail that is soluble in a fluorophilic (e.g., fluorocarbon liquid) phase, and a headgroup that is soluble in an aqueous phase.

The term “Supplementary reads” as used herein refers to an aligned part of a read already allocated a primary or a secondary aligned part.

The term “target DNA molecule” refers to a DNA molecule which comprise a specific DNA polynucleotide sequence, the “target site” or “target sequence.”

The term “Xdrop/XdropSort” as used herein refer to Xdrop- or XdropSort-system, -instrument, -sorting cartridge or -kits marketed by Samplix Aps, Birkerød, Denmark.

In particularly, “Xdrop” may be used to refer to a preferred embodiment of either the single-emulsion generating cartridge (Samplix item #CA20100) or the double-emulsion generating cartridge (Samplix item #CA10100).

### DETAILED DESCRIPTION

Current tools for characterising and verifying the long-context information of DNA modification(s) made in individual cells and cell populations include targeted enrichment of long DNA fragments (the targeted Xdrop workflow, Blondal et al., 2021). Such methods both facilitate targeted long-read sequencing, as well as being significantly more cost effective for the verification of gene editing outcome. Xdrop-targeted enrichment relies on partitioning of fragmented high molecular weight (HMW) DNA into millions of double emulsion droplets, along with PCR reagents and primers, to amplify and thereby detect a single small (˜150 bp) amplicon located within or near the region of interest, such as the site of gene editing. Droplets, containing the detected DNA of interest, are then sorted and the selected DNA then amplified using MDA to generate sufficient copies for sequencing. MDA was reported to generate long DNA molecules suitable for both short- and long-read sequencing. Furthermore, MDA was shown to generate ˜1.5 μg of amplified DNA from just 6 pg of input DNA.

Despite the reported many advantages of methods for target DNA enrichment and amplification using MDA, the present inventors have surprisingly found that MDA amplified DNA not is optimal for long-read nanopore sequencing. The reason for this incompatibility may be traced to the branched and chimeric nature of the DNA generated by MDA when using enzymes such as phi29. This phenomenon likely results from template switching during amplification. The inventors speculate that phi29-amplified DNA inhibits subsequent sequencing, for example by blocking the pores of nanopore sequencing devices. Additionally the chimeric nature of the amplified DNA increased the complexity and cost effectiveness of the sequence analysis of the target DNA.

Having identified the very existence and identity of the problem of obtaining long-read sequences when using phi29 MDA, the inventors sought to develop a new method for target enrichment and amplification of genomic DNA that would produce high fidelity long range amplified DNA molecules (without chimeras) from low amounts of starting DNA (10-15 ng or less). Such method should be capable of long-read sequencing using platforms such as long-range nanopore sequencing. Furthermore, the method should be compatible with determining the genetic outcome of various gene-editing technologies, especially gene editing events where the edited DNA is not presented as simply diploid but can have unique genetic alterations.

The present invention will now be described more fully hereinafter with reference to the accompanying drawings, in which exemplary embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout. Like elements will, thus, not be described in detail with respect to the description of each figure.

The present invention pertains to an in vitro method in which the concentration of a specific target DNA molecule is increased relative to the concentration of total DNA in a sample, by encapsulating the sample into multiple droplets of an emulsion, each of which containing reagents for detection of a specific target, followed by the detection of the specific target sequence within the droplets and, the physical sorting of droplets containing the target sequence. By this method a significant enrichment of DNA molecules comprising the specific region of interest is obtained.

A preferred embodiment of the invention is the method presented in method in FIG. 1. In this embodiment the general amplification of step (g) is performed by a long range PCR and employing PCR primers for annealing to the adaptors of adaptor-ligated DNA molecules.

Surprisingly, this method results in sequence reads, obtainable from nanopore sequencing, that are both longer and more numerous when compared to hitherto preferred indirect sequence capture technology for enrichment of long genomic DNA illustrated in FIG. 2, example 2 and in Blondal et al (2021).

From the data presented in FIG. 4 is clear that the “apex of the distribution of the primary reads” is 5 kb or more and significantly larger than the apex of the similar distribution of reads obtained when the general amplification is performed by the MDA technique. This is surprising because MDA in considered to replicated DNA with high fidelity and result in large fragment size (10-20 kb) (Zhou et al. (2020) Micromachines 11, 645)

The method of the present invention also provide relatively longer, uninterrupted parts of the reads that align to the reference sequence. As illustrated in FIG. 8, the ratio of bases in the mapped part of the primary aligned reads being in the range of 0-20 kb long over bases in the primary aligned reads of the same length is 0.7 or larger, preferably larger than 0.8 or ever larger than 0.9. Such a high fraction of long, uninterrupted parts of the reads that align to the reference sequence is of particular interest for analyses directed to determining the outcome of various gene editing procedures such as CRISPR-Cas9 mediated editing and characterizing CAR-T cassette integration patterns.

It is well demonstrated that encapsulating of the general amplification mixture and performing the amplification reaction in multiple droplets effectively decreases non-uniformity and amplification bias of amplification. Accordingly, in one embodiment the general amplification of the adaptor-ligated DNA molecules in step (g) is performed in droplets forming a second emulsion.

In the preferred embodiment described in example 1 the population of DNA molecules obtained in step (b) have an average size of from 10 to 12 kb. This fragmentation is obtained by passing the liquid sample of mixed DNA molecules through a shearing orifice by centrifugal force. But the fragmentation may be obtained by other procedures as well. It is also contemplated that creating a population of DNA molecules that have an average size significant different from 10 to 12 kb, e.g. from 12 to 30 kb or even larger in certain embodiments would create even larger primary reads.

In order to obtain an efficient ligation of adaptors to the DNA molecules it is preferred to make the population of DNA molecules blunt and subsequently phosphorylate the DNA molecules at the 5′ end. Typically also dA-tails are attached at the 3′ends of the DNA molecules followed by ligation of adaptors with 3′ dT overhangs to the ends of the DNA molecules.

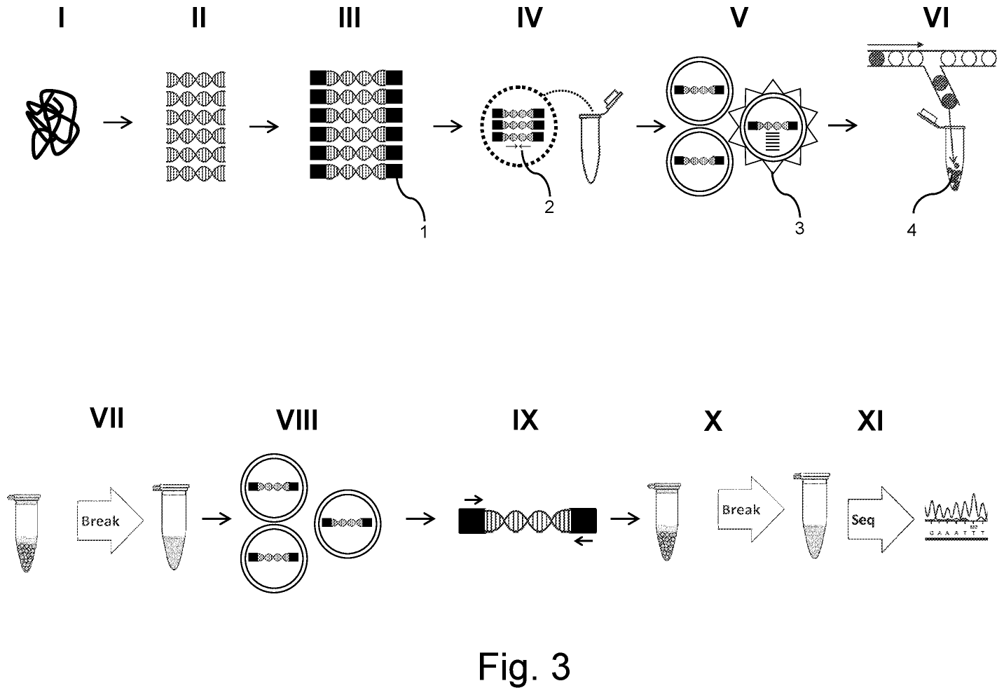

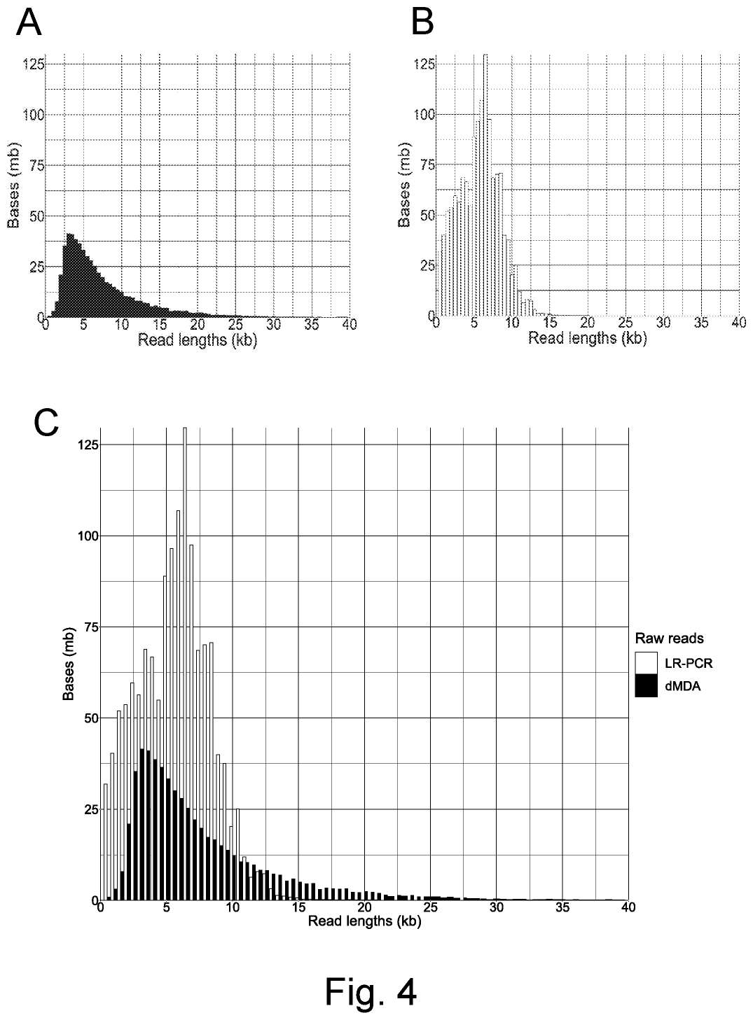

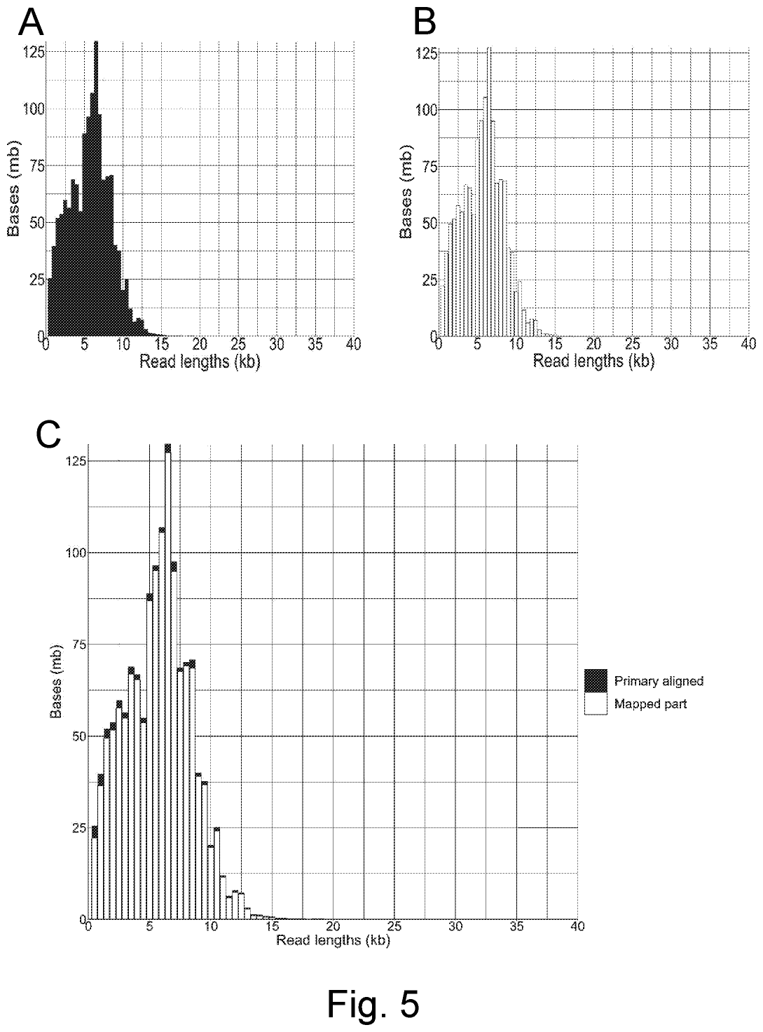

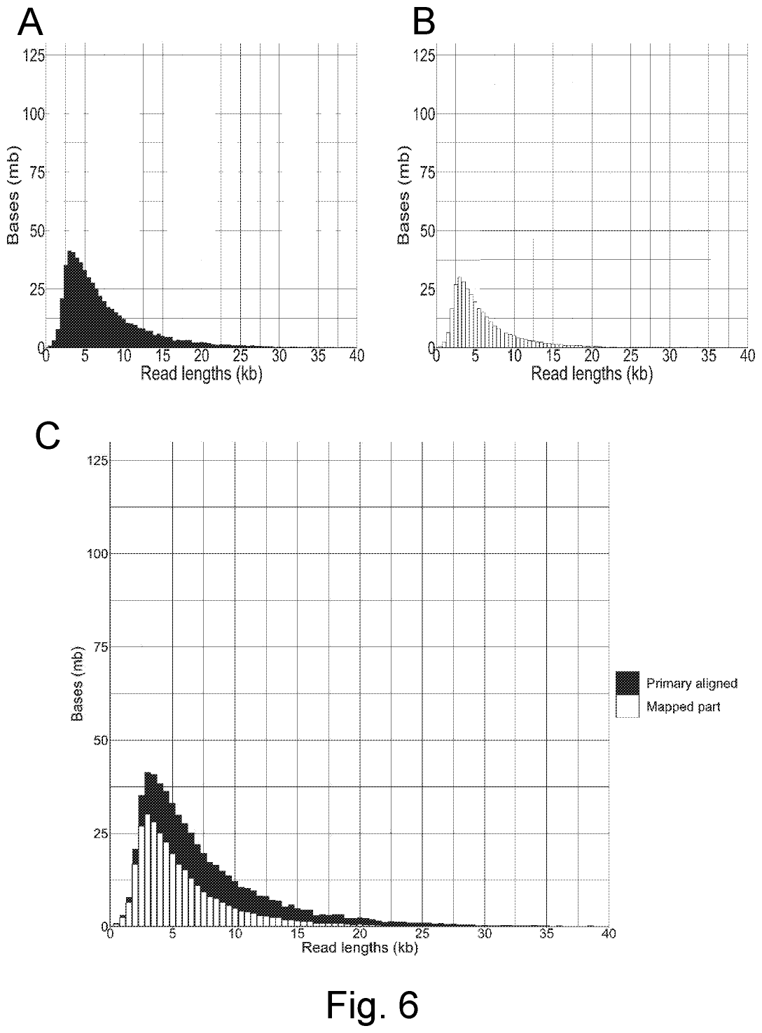

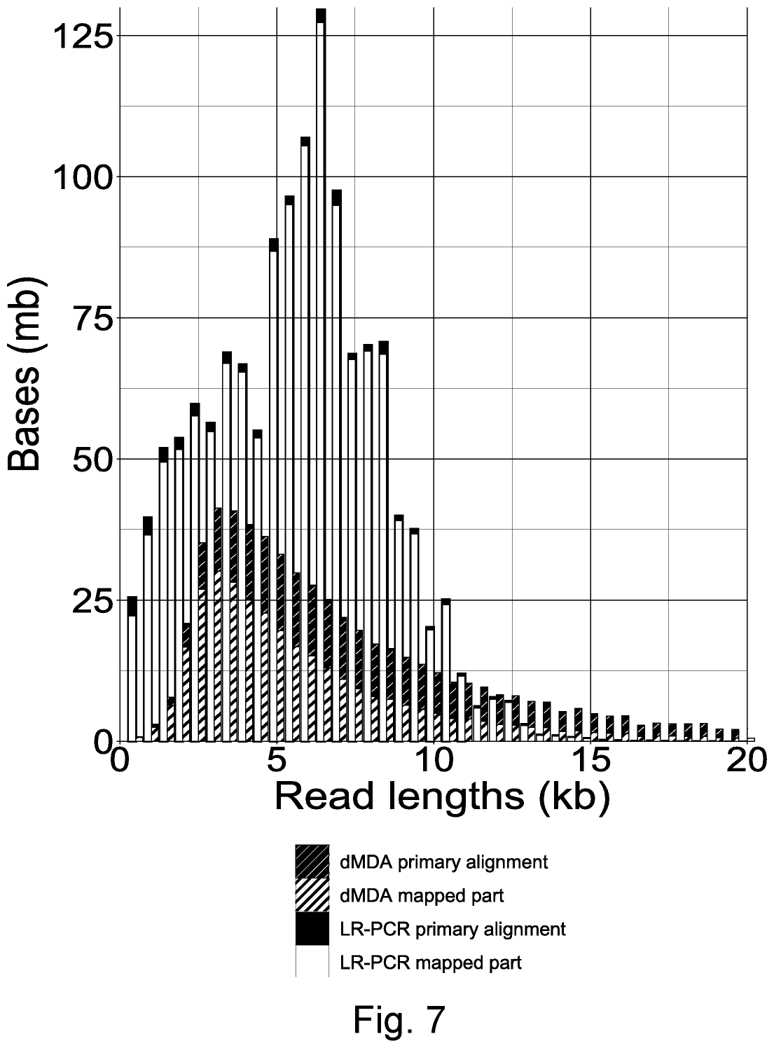

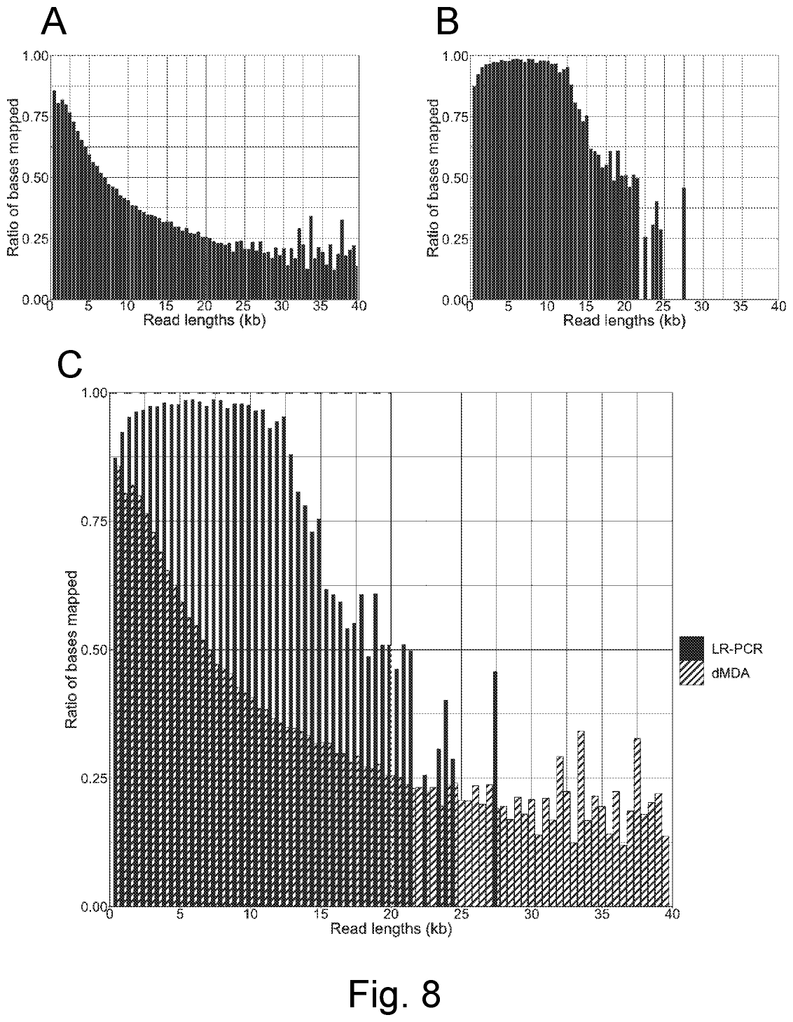

It has previously been reported that partitioning the DNA molecules and amplification reactants, e.g. of a PCR-amplification, into a plurality of smaller partitions, e.g. droplets, minimise amplification bias reported to occur during bulk amplification (EP3.314.012; Nishikawa et al. (2015) PLOS ONE | DOI: 10.1371/). Accordingly, it may be preferred to perform the general amplification step (g) on a reaction mix encapsulated in droplets. The droplets may be single- or, illustrated in FIG. 3, double-emulsion droplets. This embodiment is illustrated in FIG. 3.

It is well established that the process of ligating adaptors to the ends of the population of DNA molecules result in a population of fragments comprising both fragments with the adaptor ligated to both ends as well as fragments with the adaptor ligated to one end only and fragments without any adaptor at all.

The fraction of DNA molecules with adaptors ligated to both ends may by increased if the DNA molecules having adaptors ligated to the ends of the DNA molecules is subjected to a few, e.g. 4-8, cycles of PCR with primers directed to the adaptors, before encapsulation in double emulsion droplets. It is contemplated that such a preparatory PCR in will increase the sensitivity of the method significantly.

A further improvement to the method would be to use adaptors in step (c) that comprise barcode-sequences since it may allow multiplexing in the later steps of the procedure.

It is realized that it may be advantageous to add one or more steps wherein non-ligated adaptors remaining in the reaction mix are removed before proceeding to the next step in the method.

In particular removing non-ligated adaptors remaining on completion of step (c) prior to step (d) is thought to improve the outcome of the method. Non integrated adaptors which typically are significantly smaller than 10 bp can be removed by size exclusion spin columns, a clean-up step with beads, gel purification or even by precipitation with ethanol and 2M ammonium acetate.

The specific detection of droplets containing at least one of said target DNA molecules is typically performed by detecting a specific motif or sequence of said target DNA molecule comprising a unique consecutive sequences of at least 40 nucleotides. It is preferred that the reagents for specific detection of droplets containing the target DNA molecules are added to the liquid sample obtained in step (c).

The actual detection of the target DNA can in principle be accomplished by one of many hybridization assays base on labelled sequence specific probes. The use of e.g. an assay based on molecular beacons Tyagi et al (1996).

In order to obtain sufficiently strong signals from the positive droplets PCR-based methods are preferred. The PCR-based detection may be based on the so-called TaqMan-technique (U.S. Pat. No. 6,485,903B1) or on the staining of a specific amplified short DNA sequence with a DNA-binding fluorescent dye. In either case the reagents are added to the liquid sample obtained in step (c) and the specific detection of droplets containing at least one of the target DNA molecules in step e) is performed by the PCR reaction.

However, whether the specific detection of positive droplets involves PCR or not, a method involving nucleic acid hybridisation with fluorophore-labelled probes is preferred.

To obtain the enrichment, the positively detected droplets are sorted from the negative non-detected droplets in a process that typically involves the physical sorting of droplets containing at least one of said target DNA molecules in a step which is performed using a fluorescence-activated cell sorting device (FACS) or a microfluidic droplet sorting device. One such microfluidic droplet sorting device is described in PCT/EP2021/083518.

The aspect of target enrichment of target DNA is of special concerns. In general the fewer target molecules that on average are encapsulated in a droplet the higher enrichment is obtained by the sorting step (WO2016207379A1). Accordingly in one embodiment of the method the droplets of the double-emulsion of step (d) on average comprise very few or even less than one target DNA molecule per droplet.

In the event that the general amplification of the adaptor-ligated DNA molecules in step (g) is performed in droplets forming a second emulsion the droplets of the second emulsion are subsequently coalesced. This may conveniently be accomplished using the Break solution and Break colour from the dPCR kit (Samplix, cat no. RE10100) according to manufacturer recommendations.

The Xdrop system marketed by Samplix ApS (Birkerød, Denmark) provides a highly efficient, system for creating either single- or double emulsion droplets depending on the type of droplet-forming cartridge inserted into the Xdrop instrument. This system may provide very high numbers of droplets.

Obviously, the more droplets formed during step c) and sorted in step e) the more positive droplets may be obtained. In one preferred embodiment of the in vitro method, the total number of droplets formed in step d) is at least 5×10⁵.

Similarly if the general amplification step is performed in droplets forming a second emulsion the yield of the amplication very much depends on the number of droplets in the emulsion. Accordingly, in one further preferred embodiment of the in vitro method, the general amplification is performed on at least 1.2×10⁶ and up to a maximum of 1.2×10⁹ droplets pr. each 5 ml of the reaction mixture.

It will be appreciated, that the functionality of the invention is critically dependent on the actual microfluidic device and the reactants used. Accordingly, a kit of parts for carrying out the method is provided.

In one preferred embodiment the kit of parts comprises one or more microfluidic devices (cartridges) to form the double-emulsion droplets of step d) and optionally for the second emulsion of step (g); adaptors suitable to perform step c) of ligating adaptors to the ends of the population of DNA molecules; vials of a suitable oil composition comprising a suitable surfactant and the necessary buffers to form the emulsions of step d) and optionally of the second emulsion of step (g); vials of a suitable breakage solution and a suitable buffer/dye to rescue the DNA in the droplets selected in step f), and optionally after the general amplification performed on the second emulsion composition; and a manual for performing the method.

The advances of the present invention over the previous dMDA method (Blondal et al. (2021)) is demonstrated in table 1, example 1 and 2 and the associated figures. Indicating the method is especially advantageous for de novo assembly of long range nanopore generated sequence reads to a contiguous assembly.

In view of the literature reporting that compared with PCR-based methods, multiple displacement amplification reduces amplification bias several orders of magnitude and generates much longer amplicons (Chen et al. (2014); Dean et al. (2002)) it is surprising that a method based on a PCR-based general amplification produces significantly more (tab 1) and larger reads (FIG. 4).

TABLE 1

| | LR-PCR | dMDA |
|---|---|---|
| Number of reads | 472201 | 132480 |
| Number of primary mapped reads | 438261 | 131643 |
| Percentage of reads primary mapped to | 92.81 | 99.37 |
| reference | | |
| Number of bases primary mapped reads | 1371165153 | 586286862 |
| Number of bases in the aligned part of | 1331546775 | 317285266 |
| the primary mapped read | | |
| Percentage of bases in the aligned part | 97.11 | 54.12 |
| of the primary mapped read | | |

### The Invention Presented in the Form of Embodiments

Preferred aspects and embodiments of the invention may be presented as items of the specification. These are given below.

1. An in vitro method for enriching for one of more target DNA molecules being DNA molecules comprising a specific motif or sequence from a sample of mixed DNA molecules, wherein the method comprises the steps of:
a) providing a liquid sample of mixed DNA molecules comprising one or more specific target DNA molecule,
b) fragmenting the mixed DNA molecules of the liquid sample to obtain a population of DNA molecules having an average size of from 5 to 40 kb,
c) ligating adaptors to the ends of the population of DNA molecules of step (b) to obtain a liquid sample of adaptor-ligated DNA molecules,
d) forming of an emulsion of a multiple of double emulsion droplets from the liquid sample obtained in step (c),
e) specifically detecting droplets containing at least one of said target DNA molecules,
f) physically sorting and coalescing droplets containing at least one of said target DNA molecules, and
g) general amplification of the adaptor-ligated DNA molecules of the selected and coalesced droplets obtained in step (f).
   2. The in vitro method of item 1, wherein the general amplification of step (g) is performed by a long range PCR and employing PCR primers for annealing to the adaptors of the adaptor-ligated DNA molecules.
   3. The in vitro method according to item 1 or 2, wherein the apex of the distribution of the primary reads is 5 kb or more.
   4. The in vitro method of any of the preceding items, wherein the ratio of bases in the mapped part of the primary aligned reads being 0-20 kb long over bases in the primary aligned reads of the same length is 0.7 or larger.
   5. The in vitro method of any of the preceding items, wherein the general amplification of the adaptor-ligated DNA molecules in step (g) is performed in droplets forming a second emulsion.
   6. The in vitro method of any of the preceding items, wherein the population of DNA molecules obtained in step (b) have an average size of from 10 to 12 kb.
   7. The in vitro method of any of the preceding items, wherein the population of DNA molecules is obtained by passing the liquid sample of mixed DNA molecules through a shearing orifice by centrifugal force.
   8. The in vitro method according to any of the preceding items, wherein the population of DNA molecules are made blunt-ended and subsequently phosphorylated at the 5′ end.
   9. The in vitro method according to item 8, wherein dA-tails are attached at the 3′ends of the DNA molecules followed by ligation of adaptors to the ends of the DNA molecules, said adaptors comprise 3′ dT overhangs.
   10. The in vitro method of item 8 or 9, wherein the DNA molecules having adaptors ligated to the ends of the DNA molecules is subjected to 4-8 cycles of PCR before forming the double emulsion droplets in step d.
   11. The in vitro method according to any of the preceding items, wherein the adaptors in step (c) comprise barcode-sequences.
   12. The in vitro method according to any of the preceding items, wherein non-ligated adaptors remaining on completion of step (c) are removed prior to step (d).
   13. The in vitro method according to any of the preceding items, wherein the specific motif or sequence of said target DNA molecule comprises a unique consecutive sequences of at least 40 nucleotides.
   14. The in vitro method according to any of the preceding items, wherein reagents for specific detection of droplets containing the target DNA molecules are added to the liquid sample obtained in step (c).
   15. The in vitro method according to any of the preceding items, wherein PCR reagents are added to the liquid sample obtained in step (c) and the specific detection of droplets containing at least one of the target DNA molecules in step e) is performed by a PCR reaction.
   16. The in vitro method according to item 14, wherein the specific detection of droplets containing at least one of said target DNA molecules in step e) is performed by nucleic acid hybridisation with fluorophore-labelled probes.
   17. The in vitro method according to item 14 or 15, wherein the reagents added to the liquid sample obtained in step (c) comprise a DNA-binding fluorescent dye.
   18. The in vitro method according to any of the preceding items, wherein the physically sorting of droplets containing at least one of said target DNA molecules in step (e) is performed using a fluorescence-activated cell sorting device (FACS) or a microfluidic droplet sorting device.
   19. The in vitro method according to any of items 5-18, wherein droplets of the double-emulsion of step (d) on average comprise less that one target DNA molecule per droplet.
   20. The in vitro method according to any of items 5-15, wherein following step (g), droplets of the second emulsion are coalesced.
   21. The in vitro method according to any of the preceding items, wherein the total number of droplets formed in step d) is at least 5×10⁵.
   22. The in vitro method according to any of items 5-21, wherein the general amplification is performed on at least 1.2×10⁶ and up to a maximum of 1.2×10⁹ droplets pr. each 5 ml of the reaction mixture.
   23. A kit of parts for carrying out the method according to any one of the preceeding items 1-17, comprising:
i) one or more microfluidic devices to form to form the double-emulsion droplets of step d) and optionally the second emulsion of step (g);
ii) adaptors suitable to perform step c) of ligating adaptors to the ends of the population of DNA molecules;
iii) vials of a suitable oil composition comprising a suitable surfactant and the necessary buffers to form the emulsions of step d) and optionally of the second emulsion of step (g);
iv) vials of a suitable breakage solution and a suitable buffer/dye to rescue the DNA in the droplets selected in step f), and optionally after the general amplification performed on the second emulsion composition;
and
v) a manual for performing the method.

### EXAMPLES

### Example 1: Improved Method for Enrichment for Target DNA Molecules in a Sample of Mixed DNA Molecules

In this example an improved method for enriching for large sized DNA molecules comprising a specific target DNA motif or sequence is demonstrated. The enriched collection of DNA molecules is particularly suitable for long-range DNA sequencing directed to the analysis of the outcome of various gene-editing technologies, e.g. to detect the genetic changes after CRISPR-Cas9 editing. The various steps of the method are illustrated in FIG. 1.

### DNA Sample Preparation.

500 ng of High Molecular Weight human DNA (Female DNA, Promega Cat #G1521) was fragmented by gTUBE (Covaris Inc. Cat. #520079) fragmentation to approx. 8-15 kb in size. The sample was DNA repaired and dA end prepped using NEBNEXT companion module (New England Biolabs Inc. Cat #E7180S). A universal adaptor was ligated onto the DNA using the PCR barcoding expansion kit 1-12 (Oxford Nanopore, Cat #EXP-PBC001) and the DNA purified using capture beads (MagBio Inc. Cat #AC-60050) according to the manufacturer's recommendations. The size of the products was analysed using Tapestation (Agilent Inc. Cat #4200).

### Genomic Region-Specific Enrichment.

To perform enrichment of DNA molecules, in this case DNA molecules that comprise the RAG2 (Recombination Activating 2 Protein) gene region, a PCR reaction mix composed of:
2 μL of 7.1 ng/μL adaptor-ligated S2 DNA molecules
25.0 μL dPCR mix (2×) (Samplix Aps)
1.0 μL 10 μM forward primer (RAG2_10 kb_D_4F, ACCTGCCAGGGTAAGATTGC)
1.0 μL 10 μM reverse primer (RAG2_10 kb_D_4R, TGATGAGCAGTAATGGGTGGT)
21.0 μL molecular grade water
   were made.

Subsequently the PCR reaction mix were encapsulated in double emulsion droplets (Water-in-Oil-in-Water) as described by Madsen et al., 2020 (Human mutation doi: 10.1002/humu.24063) and Blondal et al. (2021) Methods 191, 68-77, using an Xdrop instrument (item #IN00100, Samplix ApS, Birkerød, Denmark) and a double-emulsion generating cartridge (Samplix item #CA10100).

### PCR

The generated droplets were distributed into a 0.2 mL PCR vial and the following PCR protocol applied:

Initial warm up at 30° C. for 5 sec followed by denaturation at 94° C. for 3 min followed by 40 cycles of 94° C. for 3 sec, 60° C. for 30 sec. Then the sample was cooled down to 4° C. until the droplets were harvested.

### Sorting

After droplet PCR, the droplets were stained in 1 ml 1×dPCR buffer and 10 μl droplet dye (both available as Cat. No. RE10100, Samplix Aps, Birkerød) and incubated at room temperature for 5 min, protected from light.

The positive droplet populations were then sorted from the negative using a SONY benchtop SH800S cell sorter with a 100 μm nozzle (Sony Biotechnology). The positive green fluorescent droplets were sorted from the negative droplets and collected into 15 μl of molecular grade H₂O at the bottom of a 1.5 ml DNA LoBind collection tube. The droplets were then broken using Break Solution and Break Dye (Samplix ApS, cat. No. RE20300) according to the manufacturer recommendations.

### Long Range PCR

The aqueous volume extracted from the breaking of sorted positive droplets was adjusted to 20 uL and the long-range PCR was setup immediately using Barcode primers from the Barcoding extension kit (Oxford Nanopore, Cat #EXP-PBC001) and LongAmp Taq mastermix (New England Biolabs Inc. Cat #M0287S) according to the manufacturers recommendations and the following Long range PCR protocol applied:

Initial denaturation at 95° C. for 3 min, followed by 20 cycles of 95° C. for 15 sec, 56° C. for 15 sec and 65° C. for 12 min. Then final extension at 65° C. for 10 min.

The size of the long range PCR products were analysed using Tapestation (Agilent Inc. Cat #4200).

### Sequencing

The long-range-PCR products were capture bead purified, pooled and DNA repaired and dA end prepped by using the NEBNext® Companion Module (New England Biolabs Cat #E7180S) according to the manufacturing recommendations. Sequencing adaptors were ligated onto the library pool ends using Ligation Sequencing Kit (Oxford Nanopore, Cat #SQK-LSK109) and 5 fmol of the DNA library sequenced on a R9 Flowcell (Oxford Nanopore, Cat #R9.4.1) on a GridION instrument according to the manufacturers recommendations.

Data was basecalled by the Oxford Nanopore Guppy 5.0.17 basecalling software using the “super high accuracy” setting and with quality threshold 10.

### Results and Analysis

The FastQ sequence data files of the basecalled data was analysed by the Minimap2 software package (Li (2018)) with default parameters

The results are shown in FIGS. 4-8 and table 2.

TABLE 2

| | LR-PCR |
|---|---|
| Number of reads | 472201 |
| Number of primary mapped reads | 438261 |
| Percentage of reads primary mapped to | 92.81 |
| reference | |
| Number of bases primary mapped reads | 1371165153 |
| Number of bases in the aligned part of the | 1331546775 |
| primary mapped read | |
| Percentage of bases in the aligned part of the | 97.11 |
| primary mapped read | |

### Example 2: Enrichment for Target DNA Molecules in a Sample of Mixed DNA Molecules with Standard dMDA Amplification

To illustrate the advantages of the present invention, we made comparative data using the multiple displacement amplification in droplets (dMDA) technique for the general amplification step described by Blondal et al. (2021) Methods 191, 68-77.

### Genomic Region-Specific Enrichment.

A sample of high molecular weight human DNA-molecules (Female DNA, Promega Cat #G1521) were region-specific enriched.

To perform the region-specific enrichment of the DNA molecules, in this case DNA molecules that comprise a 125 bp part of the RAG2 (Recombination Activating 2 Protein) gene region, a PCR reaction mix composed of:
2 μL of 5 ng/μL Female DNA molecules
25.0 μL dPCR mix (2×) (Cat no. RE10100 Samplix Aps)
1.0 μL 10 μM forward primer (RAG2_10 kb_D_4F, ACCTGCCAGGGTAAGATTGC)
1.0 μL 10 μM reverse primer (RAG2_10 kb_D_4R, TGATGAGCAGTAATGGGTGGT)
21.0 μL Water
   were made.

Subsequently the PCR reaction mix were encapsulated in double emulsion droplets (Water-in-Oil-in-Water) as described by Madsen et al., 2020 (Human mutation doi: 10.1002/humu.24063) and Blondal et al. (2021) Methods 191, 68-77, using an Xdrop instrument (item #IN00100, Samplix ApS, Birkerød, Denmark) and a double-emulsion generating cartridge (Samplix item #CA10100).

The produced droplets were transferred to PCR vials and subjected to PCR using the temperature cycling conditions shown below.

TABLE 3

| Temperature | Cycle Duration | Repetitions |
|---|---|---|
| 30° C. | 5 sec | 1x |
| 94° C. | 3 min (slow ramp) | 1x |
| 94° C. | 3 sec (slow ramp) | 40x |
| 60° C. | 30 sec (slow ramp) | |
| 4° C. | Forever | 1x |

After PCR, the droplets were stained with the Droplet dye from the dPCR kit (Cat no. RE10100, Samplix Aps, Birkerød) and FACS sorted using the SH800S Cell Sorter (Sony Biotechnology) and a 100 μM nozzle as describe in Example 1.

The (1029) positive droplets were coalesced using the Break solution and Break colour from the dPCR kit (Samplix, cat no. RE10100) according to manufacturer recommendations.

The retrieved DNA was immediately used to set up a single emulsion droplet Multiple Displacement Amplification (dMDA) using the Xdrop instrument together with the Xdrop dMDA kit, dMDA cartridge, dMDA holder, and dMDA gasket (Samplix Aps Cat nos. RE20300, CA20100, HO10100, and, GA20200 respectively).

The reagents were mixed as shown in the table below and loaded into the dDMA cartridge in the dMDA holder, sealed with the dDMA gasket, and the cartridge loaded into the Xdrop instrument according to manufacturer recommendations.

TABLE 4

| Reagent | μL |
|---|---|
| dMDA mix | 4 |
| H2O | 5 |
| dMDA enzyme | 1 |
| Total mix | 10 |
| Harvested DNA | 10 |
| Total volume | 20 |

The produced droplets were transferred to PCR vials and incubated at 30° C. for 16 hours followed by enzyme inactivation at 65° C. for 10 minutes.

DNA was harvested from the dMDA droplets using the Break solution and Break colour from the dMDA kit according to manufacturer recommendations. The harvested DNA was quantified on a Quantus instrument (Promega) using the QuantFluor dsDNA system (Promega) and the size of the DNA estimated on a Tapestation 4200 instrument (Agilent). Then the DNA from the duplicate reactions was pooled and re-quantified on a Quantus instrument (Promega) using the QuantFluor dsDNA system (Promega).

The retrieved dMDA DNA, was used for construction Oxford Nanopore library and sequenced on a GridION (Oxford Nanopore Technologies) as follows: First 1100 ng of dMDA DNA was debranched for 15 minutes at 37° C. in a 50 μL reaction volume containing 1.5 μL T7 Endonuclease I (New England Biolabs), and 5 μL of 10×NEB2buffer (New England Biolabs) (NEB). The debranched DNA was then size selected by adding 35 μL of MagBio magnetic beads which was were washed with water and then custom buffered before use in 10 mM Tris-HCl pH8, 1 mM EDTA pH8, 1.6M NaCl, and 11% PEG8000 buffer.

The debranched DNA with custom buffered beads was incubated for 20 minutes at room temperature with gentle rotation. Then the beads were pelleted in the tube on a magnet and the buffer removed followed by washing twice in 200 uL 70% ethanol and complete removal of the ethanol.

The bead pellet was resuspended in 52 μL of nuclease-free water and incubated at 50° C. for 1 minute and room temperature for 5 minutes followed by pelleting of the beads on a magnet and removal of 50 μL of eluate into a clean 0.2 ml tube.

The DNA eluate was quantified on a Quantus instrument (Promega) using the QuantFluor dsDNA system (Promega) and the size of the DNA estimated on a Tapestation 4200 instrument (Agilent).

The DNA eluate was repaired and 3′-dA overhangs added using the NEBNext® Companion Module (New England Biolabs Cat #E7180S) according to the manufacturing recommendations. A barcode was ligated on to the repaired and end prepped DNA using the Native Barcoding Kit 13-24 (PCR free) (Oxford Nanopore, Cat #EXP-NBD114) followed by sequencing adaptor ligation using the Ligation Sequencing Kit (Oxford Nanopore, Cat #SQK-LSK109) according to manufacturers recommendations.

Finally, the resulting library was quantified using the Quantus instrument (Promega) using the QuantFluor dsDNA system (Promega) and 20 fmol of the DNA library was sequenced on a R9 Flowcell (Oxford Nanopore, Cat #FLO-MIN106D) on a GridION instrument (Oxford Nanopore) according to the manufacturers recommendations.

Data was basecalled by the Oxford Nanopore Guppy 5.0.17 basecalling software using the “super high accuracy” setting and with quality threshold 10.

### Results and Analysis

The FastQ sequence data files of the basecalled data was analysed by the Minimap2 software package (Li (2018) Bioinformatics. 34, 3094-3100) with default parameters

The results are shown in FIGS. 4-8 and table 5.

TABLE 5

| | dMDA |
|---|---|
| Number of reads | 132480 |
| Number of primary mapped reads | 131643 |
| Percentage of reads primary mapped to reference | 99.37 |
| Number of bases primary mapped reads | 586286862 |
| Number of bases in the aligned part of the | 317285266 |
| primary mapped read | |
| Percentage of bases in the aligned part of the | 54.12 |
| primary mapped read | |

### REFERENCES

Blanco et al., (1989) Highly efficient DNA synthesis by the phage phi 29 DNA polymerase. Chem. 264: 8935-40.
Blondal et al., (2021) Verification of CRISPR editing and finding transgenic inserts by Xdrop indirect sequence capture followed by short- and long-read sequencing; Methods 191, 68-77.
Chen et al. (2014) Comparison of Multiple Displacement Amplification (MDA) and Multiple Annealing and Looping-Based Amplification Cycles (MALBAC) in Single-Cell Sequencing. PLOS One. 9(12): e114520.
Dean et al., (2002) Comprehensive human genome amplification using multiple displacement amplification Proc Natl Acad Sci USA. 99(8): 5261-5266.
Li (2018), Minimap2: pairwise alignment for nucleotide Sequences. Bioinformatics. 34, 3094-3100.
Madsen et al., (2020) Xdrop: Targeted sequencing of long DNA molecules from low input samples using droplet sorting Human Mutation. 2020; 41:1671-1679.
Nishikawa et al. (2015) Monodisperse Picoliter Droplets for Low-Bias and Contamination-Free Reactions in Single-Cell Whole Genome Amplification. PLOS One. 10(9): e0138733.
Steele, et al., (2020) Novel CRISPR-based sequence specific enrichment methods for target loci and single base mutations PLOS One. 2020; 15(12)
Tyagi et al. (1996), Molecular Beacons: Probes that Fluoresce upon Hybridization. Nat Biotechnol 14:303-308.
Zanoli et al. (2013) Isothermal Amplification Methods for the Detection of Nucleic Acids in Microfluidic Devices Biosensors 3, 18-43.
Zhou et al. (2020) Micromachines 11, 645.

1. An in vitro method for enriching one or more target DNA molecules being wherein said DNA molecules comprise a specific motif or sequence from a sample of mixed DNA molecules, wherein the method comprises the steps of:
a) providing a liquid sample of mixed DNA molecules comprising one or more specific target DNA molecules,
b) fragmenting the mixed DNA molecules of the liquid sample to obtain a population of DNA molecules having an average size of from 5 to 40 kb,
c) ligating adaptors to the ends of the population of DNA molecules of step (b) to obtain a liquid sample of adaptor-ligated DNA molecules,
d) forming an emulsion of a multiple of double emulsion droplets from the liquid sample obtained in step (c),
e) specifically detecting droplets containing at least one of said target DNA molecules,
f) physically sorting and coalescing droplets containing at least one of said target DNA molecules, and
g) general amplification of the adaptor-ligated DNA molecules of the selected and coalesced droplets obtained in step (f).

2. The in vitro method of claim 1, wherein the general amplification of step (g) is performed by a long range PCR and employing PCR primers for annealing to the adaptors of the adaptor-ligated DNA molecules.

3. The in vitro method according to claim 2, further comprising step (h) sequencing products of general amplification obtained in step g), wherein the apex of the distribution of primary reads obtained by said sequencing is 5 kb or more.

4. The in vitro method of claim 3, wherein the ratio of bases in the mapped part of the primary aligned reads being 0-20 kb long over bases in the primary aligned reads of the same length is 0.7 or larger.

5. The in vitro method of claim 1, wherein the general amplification of the adaptor-ligated DNA molecules in step (g) is performed in droplets forming a second emulsion.

6. The in vitro method of claim 1, wherein the DNA molecules having adaptors ligated to the ends of the DNA molecules in step c) subsequently are subjected to 4-8 cycles of PCR before forming the double emulsion droplets in step d.

7. The in vitro method according to claim 1, wherein PCR reagents are added to the liquid sample obtained in step (c) and the specific detection of droplets containing at least one of the target DNA molecules in step e) is performed by a PCR reaction.

8. The in vitro method according to claim 1, wherein the physically sorting of droplets containing at least one of said target DNA molecules in step e) is performed using a fluorescence-activated cell sorting device (FACS) or a microfluidic droplet sorting device.

9. The in vitro method according to claim 5, wherein droplets of the double-emulsion of step (d) on average comprise less that than one target DNA molecule per droplet.

10. A kit of parts for carrying out the method according to claim 1, comprising:
I) one or more microfluidic devices to form to form the double-emulsion droplets of step d) and optionally the second emulsion of step (g);
ii) adaptors suitable to perform step c) of ligating adaptors to the ends of the population of DNA molecules;
iii) vials of a suitable oil composition comprising a suitable surfactant and the necessary buffers to form the emulsions of step d) and optionally of the second emulsion of step (g);
iv) vials of a suitable breakage solution and a suitable buffer/dye to rescue the DNA in the droplets selected in step f), and optionally after the general amplification performed on the second emulsion composition;
and
v) a manual for performing the method.
